(19) United States
(12) Patent Application Publication
(10) Pub. No.: US 2016/0143755 A1
(43) Pub. Date: May 26, 2016

(54) SELF-EXPANDABLE STENT

(71) Applicant: ASAHI INTECC CO., LTD., Nagoya-shi (JP)

(72) Inventor: Makoto NISHIGISHI, Owariasahi-shi (JP)

(21) Appl. No.: 14/842,094

(22) Filed: Sep. 1, 2015

(30) Foreign Application Priority Data
Nov. 26, 2014 (JP) 2014-238858

(51) Int. Cl. A61F 2/848; A61F 2/844; A61F 2/88

(52) U.S. Cl. CPC A61F 2/848; A61F 2/885; A61F 2/844; A61F 2002/8486; A61F 2002/823

(57) ABSTRACT

A self-expandable stent includes a plurality of first wires and a plurality of second wires interwoven together. A first anchor member is attached to a distal end of one of the plurality of first wires. The distal end of the first anchor member is fixed to the distal end of the one of the plurality of first wires, but the proximal end is a free end and is not fixed to any of the plurality of first wires or to any of the plurality of second wires. Consequently, the self-expandable stent is capable of contacting a blood vessel wall or digestive organ wall at the optimal position when the self-expandable stent is expanded at the target site. A gap is not easily formed between the anchor member and the blood vessel wall or digestive organ wall, and thus the self-expandable stent is not easily dislocated from the target site.

### CROSS REFERENCE TO RELATED APPLICATION

This application claims priority to Japanese Application No. 2014-238858 filed on Nov. 26, 2014, the content of which is incorporated by reference herein in its entirety.

### BACKGROUND

The disclosed embodiments relate to a medical device. Specifically, the disclosed embodiments relate to a self-expandable stent for supporting the lumen of a blood vessel or digestive organ in order to prevent restenosis of a narrowed or obstructed segment formed in the blood vessel or digestive organ, or for confining an embolization coil in an aneurysm formed in an arterial vessel of the abdomen or the brain to prevent the aneurysm from rupturing.

Stents are medical devices used for treating a stenosis or occlusion or for treating an aneurysm formed in a blood vessel or digestive organ (hereinafter referred to as a “target site”). Stents are broadly classified into two categories: a balloon-expandable stent which is expandable with a balloon catheter, and a self-expandable stent in which the stent expands spontaneously. Recently, self-expandable stents have often been used that are resistant to deformation even when an external force is applied.

In the case of a self-expandable stent, the stent is usually released at the target site from the distal end of a catheter by pushing a pusher guide wire in the direction of the distal end after delivering the catheter to the target site. In order to prevent dislocation of the stent from the target site during expansion, an anchor member may be attached to an end of the self-expandable stent (for example, see JP-A-2014-513987 and JP-B2-4672241). The anchoring effect of an anchor member on the wall of a blood vessel or digestive organ can prevent the self-expandable stent from moving axially or rotating radially when the self-expandable stent is expanded at the target site.

However, in the self-expandable stents according to JP-A-2014-513987 and JP-B2-4672241, the anchor member does not have a degree of freedom with respect to the wire of the stent on which it is mounted, since both the distal end and the proximal end of the anchor member are fixed to the wire. Therefore, the anchor member cannot easily contact the blood vessel wall or digestive organ wall at the optimal position when the self-expandable stent is expanded at the target site. As a result, the anchor effect of the anchor member on the blood vessel wall or digestive organ wall is insufficient, and thus the self-expandable stent may be disadvantageously dislocated from the target site. In particular, a gap between the anchor member and the blood vessel wall or digestive organ wall may be formed when the target site is located at a curved portion of the blood vessel or digestive organ, resulting in a significant problem in which the anchor effect of the anchor member on the blood vessel wall or digestive organ wall is insufficient.

### SUMMARY

The disclosed embodiments were devised in view of the above-discussed circumstances. An object of the disclosed embodiments is to provide a self-expandable stent in which an anchor member is capable of contacting a blood vessel wall or digestive organ wall at the optimal position, and a gap is not easily formed between the anchor member and the blood vessel wall or digestive organ wall. As a result, the self-expandable stent is not easily dislocated from the target site when the self-expandable stent is expanded at the target site.

The above objects can be achieved by the means listed below.

A self-expandable stent of the disclosed embodiments comprises a plurality of first wires; a plurality of second wires interwoven with the plurality of first wires; and a first anchor member attached to a distal end of one of the plurality of first wires. A distal end of the first anchor member is fixed to the one of the plurality of first wires, while a proximal end of the first anchor member is a free end and is not fixed to the one of the plurality of first wires.

In this manner, the first anchor member has a degree of freedom with respect to the one of the plurality of first wires, and the proximal end of the first anchor member can pivot about the distal end (which forms a fulcrum). Therefore, when the self-expandable stent is expanded at the target site, the first anchor member can easily contact the blood vessel wall or digestive organ wall at the optimal position. In particular, in a case where the target site is located at a curved portion of the blood vessel or digestive organ, the first anchor member can contact the blood vessel wall or digestive organ wall at the optimal position depending on the curvature of the blood vessel wall or digestive organ wall. Therefore, a gap is not easily formed between the anchor member and the blood vessel wall or digestive organ wall. As a result, the anchor effect of the first anchor member on the blood vessel wall or digestive organ wall can be improved, reducing a risk that the self-expandable stent will be axially or radially dislocated from the target site.

The self-expandable stent may further comprise a second anchor member attached to a distal end of one of the plurality of first and second wires that is different from the one of the plurality of first wires to which the first anchor member is attached (that is, a “different wire”). A distal end of the second anchor member is fixed to the different wire, while a proximal end of the second anchor member is a free end that is not fixed to the different wire. The second anchor member is disposed at a position different from the first anchor member in the axial direction. The anchor effect on a blood vessel wall or digestive organ wall can be improved in the axial direction by disposing the second anchor member having a degree of freedom at an axially different position in addition to the first anchor member, reducing a risk that the self-expandable stent is axially or radially dislocated from the target site.

Furthermore, a tensile strength of each of the plurality of first wires may be higher than a tensile strength of each of the plurality of second wires. The first anchor member attached to the one of the plurality of first wires with high tensile strength can therefore preferentially contact the blood vessel wall or digestive organ wall with respect to the plurality of second wires with low tensile strength when the self-expandable stent is expanded at the target site. Damage to the blood vessel wall or digestive organ wall by the distal end of each of the plurality of second wires to which the anchor member is not attached can therefore be reduced while maintaining the anchor effect of the first anchor member on the blood vessel wall or digestive organ wall.

Additionally, a center of the one of the plurality of first wires may be displaced from a center of the first anchor member, as viewed in a horizontal cross section orthogonal to the axial direction of the one of the plurality of first wires. That is, the center of the one of the plurality of first wires may be offset with respect to the center of the first anchor member. The degree of freedom of the first anchor member becomes even larger, and the range of motion of the proximal end of the first anchor pivoting with respect to the distal end can be increased. Therefore, even in a case where a blood vessel wall or digestive organ wall has an uneven surface due to the presence of a plaque or the like, contact can be made at the optimal position depending on the uneven surface. As a result, a risk can be further reduced that a self-expandable stent will be axially or radially dislocated from the target site regardless of the conditions of a blood vessel wall or digestive organ wall at the target site.

### BRIEF DESCRIPTION OF THE DRAWINGS

FIG. 1 shows an overall view of a self-expandable stent according to the disclosed embodiments.

FIG. 2 shows an enlarged view of Section A in FIG. 1.

FIG. 3 shows a self-expandable stent stored inside a catheter that is delivered near the target site.

FIG. 4 shows a self-expandable stent released at the target site to contact a blood vessel wall or digestive organ wall.

FIG. 5 shows an overall view of a self-expandable stent according to the disclosed embodiments.

FIG. 6 shows an enlarged view of a portion of a self-expandable stent according to the disclosed embodiments.

FIG. 7A shows an enlarged view of a portion of a self-expandable stent according to the disclosed embodiments. FIG. 7B is a cross-sectional view taken along line B-B of FIG. 7A.

FIG. 8 shows an overall view of a self-expandable stent according to the disclosed embodiments.

### DETAILED DESCRIPTION OF EMBODIMENTS

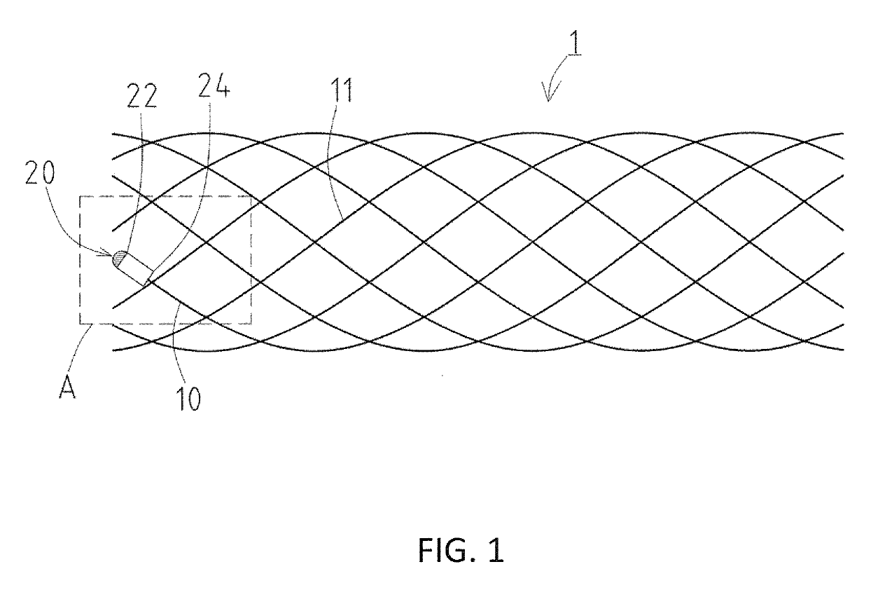

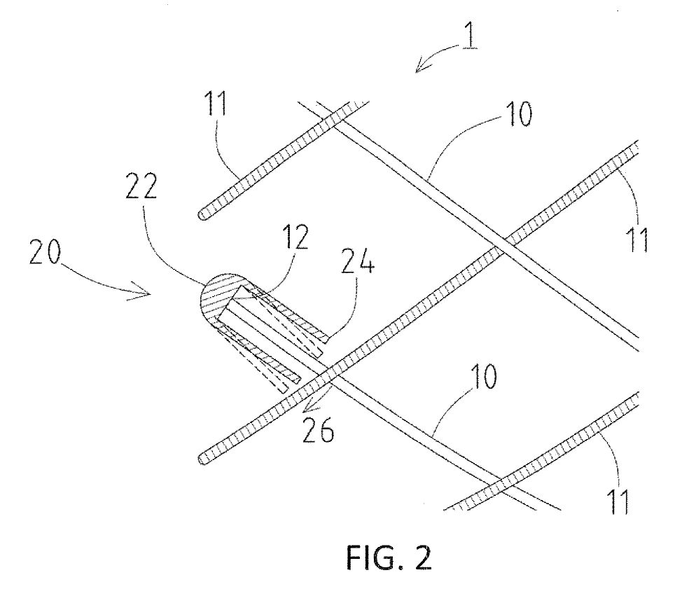

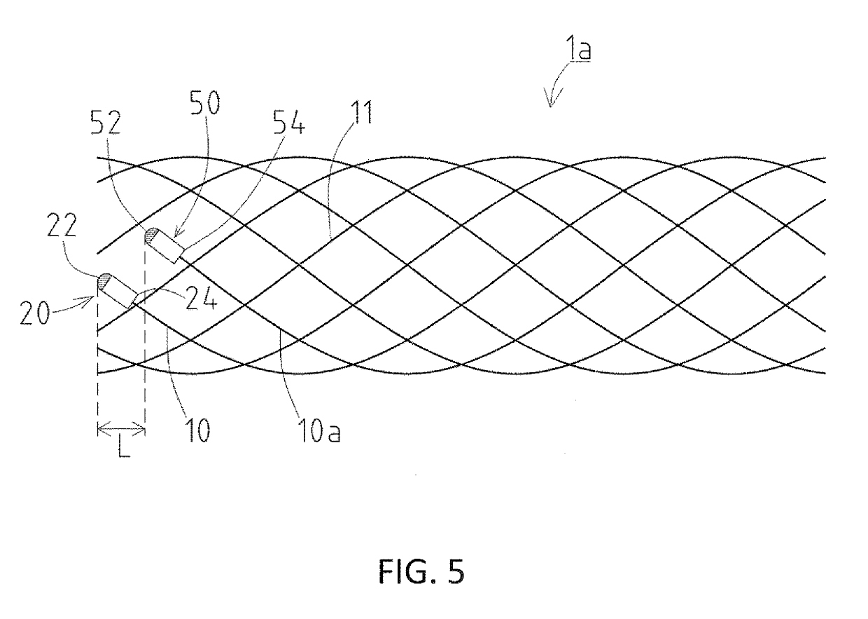

A self-expandable stent 1 according to the disclosed embodiments is described with reference to FIGS. 1 to 4. The left side in FIGS. 1 to 4 (and FIGS. 5 to 7A and 8) corresponds to the distal end (the front end), which is to be inserted into the body, and the right side corresponds to the proximal end (the base end), which is to be operated by an operator such as a physician. Note that FIG. 2 is an enlarged view of Section A in FIG. 1.

The self-expandable stent 1 is used, for example, to treat a target site formed at a blood vessel wall or digestive organ wall. As shown in FIGS. 1 and 2, the self-expandable stent 1 comprises a plurality of first wires 10 and a plurality of second wires 11 interwoven with the plurality of first wires 10 to form a mesh-like (a net-like) structure.

In the self-expandable stent 1, the plurality of first wires 10 and the plurality of second wires 11 may be made of a high tensile strength material, such as a Co-Cr alloy. For example, the plurality of first wires 10 and the plurality of second wires 11 may be made of a metal material such as stainless steel, W, Pt, a Pt—Ni alloy, a Ni—Ti alloy, or a Cu—Al—Ni alloy; or may be made of a resin material such as polyester, polyurethane, polyolefin, polytetrafluoroethylene, or a silicone resin.

Further, in the self-expandable stent 1, eight first wires 10 wind in the clockwise direction, and eight second wires 11 wind in the counterclockwise direction, and thus a total of 16 (8+8) wires 10, 11 are interwoven with each other. However, the number of the wires 10, 11 in the self-expandable stent 1 is not be limited to 16 (8+8) in total, but may be, for example, 24 (12+12) in total or 32 (16+16) in total.

As shown in FIG. 2, a single first anchor member 20 is attached to a distal end 12 of one of the plurality of first wires 10. A distal end 22 of the first anchor member 20 is fixed to the distal end 12 of the one of the plurality of first wires 10, but a proximal end 24 of the first anchor member 20 is a free end and is not fixed to the one of the plurality of first wires 10 (or any other component of the stent). Therefore, the first anchor member 20 has a degree of freedom with respect to the one of the plurality of first wires 10, and, for example, the proximal end 24 can pivot about the distal end 22 like a pendulum in the direction of an arrow 26.

As shown in FIG. 2, the first anchor member 20 is a tube body made of the same material as the first wires 10. However, there is no particular limitation for the material of the first anchor member 20, and a material different from that of the first wires 10 may alternatively be used. Further, the first anchor member 20 may be formed into a hollow coil body.

In the self-expandable stent 1, the first anchor member 20 is attached to the wire by positioning a hollow cylinder body onto the distal end 12 of the one of the plurality of first wires 10, and then irradiating the distal end 12 of the one of the plurality of first wires 10 and the distal end of the hollow cylinder body with a laser. However, the attachment method is not limited to this, and the first anchor member 20 may be attached to the distal end 12 of the one of the plurality of first wires 10 with, for example, an adhesive.

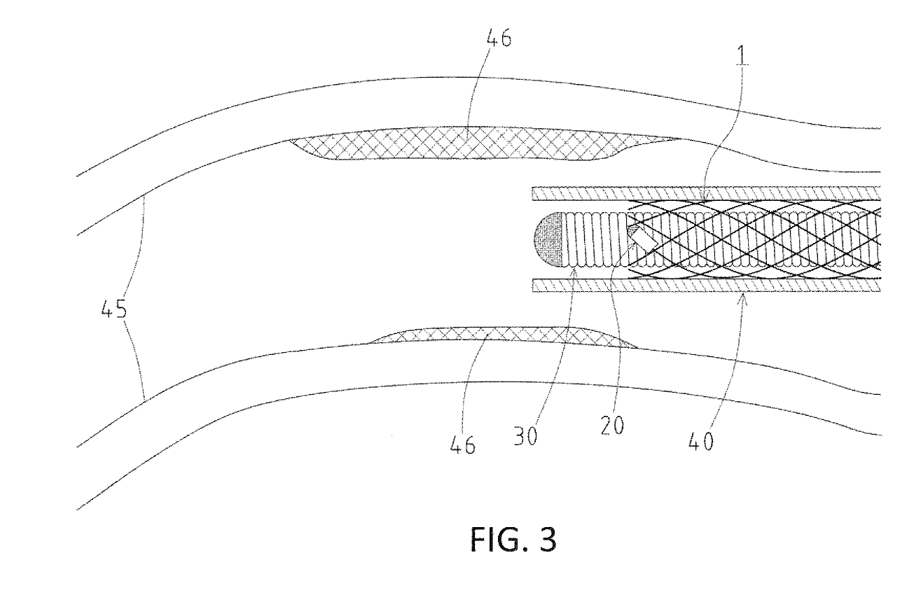

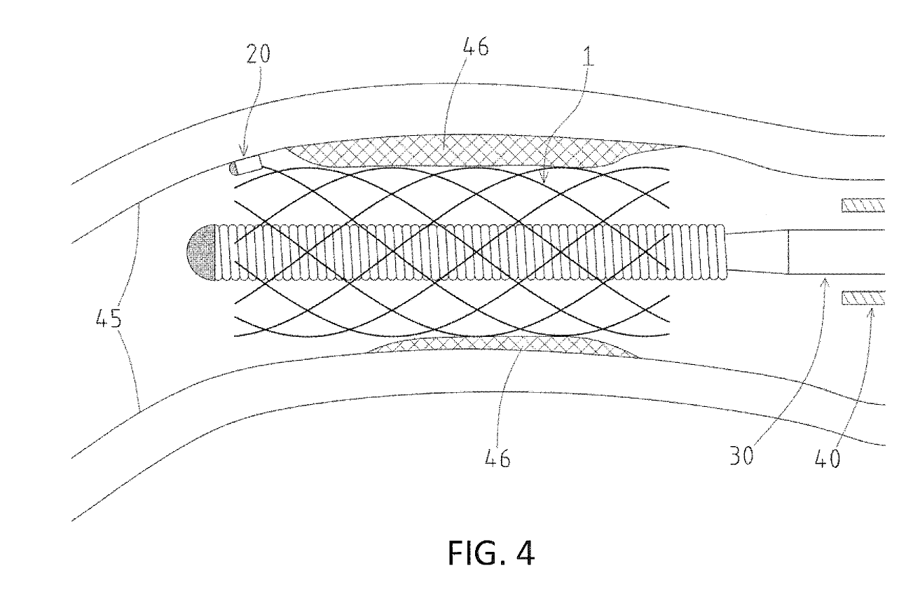

As shown in FIG. 3, the self-expandable stent 1 stored inside a catheter 40 is delivered to the near side of the target site 46 formed on a blood vessel wall or digestive organ wall 45 (that is, the side closest to the point of the initial entrance of the catheter 40 into the blood vessel or digestive organ). Then, as shown in FIG. 4, the self-expandable stent 1 is released from the distal end of the catheter 40 to the target site 46 by pushing a pusher guide wire 30 in the distal direction while pulling the catheter 40 in the proximal direction. The self-expandable stent 1 can thereby support the lumen of the blood vessel or digestive organ to prevent restenosis of the target site 46 formed on the blood vessel wall or digestive organ wall 45.

The first anchor member 20 has a degree of freedom of motion with respect to the one of the plurality of first wires 10. When the self-expandable stent 1 is expanded at the target site 46, the proximal end 24 of the first anchor member 20 can pivot about the distal end 22, and the first anchor member 20 can therefore contact the blood vessel wall or digestive organ wall 45 at the optimal position. Even in a case where the target site 46 is located at a curved portion, the first anchor member 20 can contact the blood vessel wall or digestive organ wall 45 at the optimal position depending on the curvature of the blood vessel wall or digestive organ wall 45. Therefore, a gap is not easily formed between the first anchor member 20 and the blood vessel wall or digestive organ wall 45. As a result, the anchor effect of the first anchor member 20 on the blood vessel wall or digestive organ wall 45 can be improved, reducing a risk that the self-expandable stent 1 will be axially or radially dislocated from the target site 46.

In the self-expandable stent 1 shown in FIGS. 1 to 4, a single first anchor member 20 is attached to the distal end 12 of one of the plurality of first wires 10, but the configuration is not limited to this. For example, a first anchor member 20 may be attached to a distal end of one of the plurality of second wires 11, or a plurality of first anchor members 20 may be attached to each distal end of some of the plurality of first wires 10 or the plurality of second wires 11. In the latter configuration, the plurality of first anchor members 20 may be disposed at a same position in the axial direction (the longitudinal direction of the stent). That is, the plurality of first anchor members 20 may be aligned with each other in the axial direction of the stent.

Next, a self-expandable stent la according to the disclosed embodiments is described with reference to FIG. 5. Only differences from the self-expandable stent 1 shown in FIGS. 1 to 4 will be described.

As shown in FIG. 5, in the self-expandable stent la, a second anchor member 50 is attached to a distal end (not shown) of another one of the plurality of first wires 10: a different wire 10a. The different wire 10a is different from the one of the plurality of first wires 10 to which the first anchor member 20 is attached. A distal end 52 of the second anchor member 50 is fixed to the different wire 10a, while a proximal end 54 is a free end and is not fixed to the different wire 10a (or any other component of the stent). Therefore, the second anchor member 50 has a degree of freedom of motion with respect to the different wire 10a, just as the first anchor member 20 has a degree of freedom of motion with respect to the one of the plurality of first wires 10. For example, the proximal end 54 can pivot about the distal end 52 like a pendulum.

The second anchor member 50 is disposed at a position different from the first anchor member 20 in the axial direction (the longitudinal direction of the stent). As shown in FIG. 5, the second anchor member 50 is separated by a distance L toward the proximal end relative to the first anchor member 20. As described above, in the self-expandable stent 1a, the second anchor member 50 having a degree of freedom is disposed at an axially different position in addition to the first anchor member 20. Therefore, the anchor effect on a blood vessel wall or digestive organ wall can be improved in the axial direction of the stent, further reducing a risk that the self-expandable stent 1a will be axially dislocated from the target site.

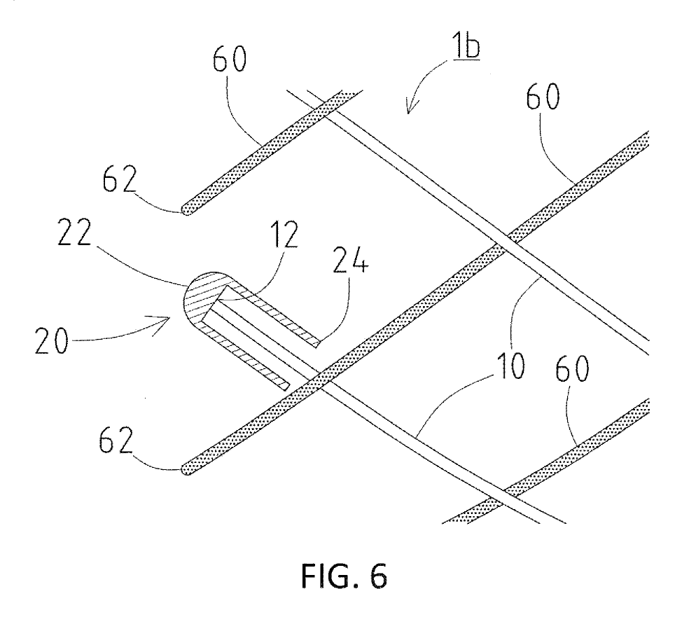

Next, a self-expandable stent 1b according to the disclosed embodiments is described with reference to FIG. 6. Note that FIG. 6 shows an enlarged view of a portion of the self-expandable stent 1b. Only differences from the self-expandable stent 1 shown in FIGS. 1 to 4 will be described.

As shown in FIG. 6, in the self-expandable stent 1b, the plurality of first wires 10 with high tensile strength are interwoven with a plurality of second wires 60 with low tensile strength to form a mesh-like (net-like) structure. That is, the tensile strength of the plurality of first wires 10 is higher than that of the plurality of second wires 60. The first anchor member 20 is attached to the distal end 12 of the one of the plurality of first wires 10 with high tensile strength, rather than a distal end 62 of one of the plurality of second wires 60 with low tensile strength.

When the self-expandable stent lb is expanded at the target site, the first anchor member 20 attached to the one of the plurality of first wires 10 with high tensile strength can preferentially contact the blood vessel wall or digestive organ wall with respect to the plurality of second wires 60 with low tensile strength. This can reduce the risk of any potential damage to the blood vessel wall or digestive organ wall by the distal end 62 of each of the plurality of second wires 60 to which the anchor member 20 is not attached, while maintaining the anchor effect of the first anchor member 20 on the blood vessel wall or digestive organ wall.

In the self-expandable stent 1b, the plurality of first wires 10 may be made of a Co—Cr alloy with high tensile strength, and the plurality of second wires 60 may be made of a Pt—Ni alloy with low tensile strength. Since the Pt—Ni alloy has radiopacity, an operator such as a physician can precisely detect the position of the self-expandable stent lb using X-ray imaging.

Note that in the self-expandable stent 1b, the tensile strength of the plurality of first wires 10 is higher than that of the plurality of second wires 60 because a metal material of the plurality of first wires 10 has higher tensile strength than that of the plurality of second wires 60. However, the materials used for the plurality of first wires 10 and the plurality of second wires 60 are not limited to metal materials, and resin materials may be used as long as the tensile strength of the plurality of first wires 10 will be higher than that of the plurality of second wires 60.

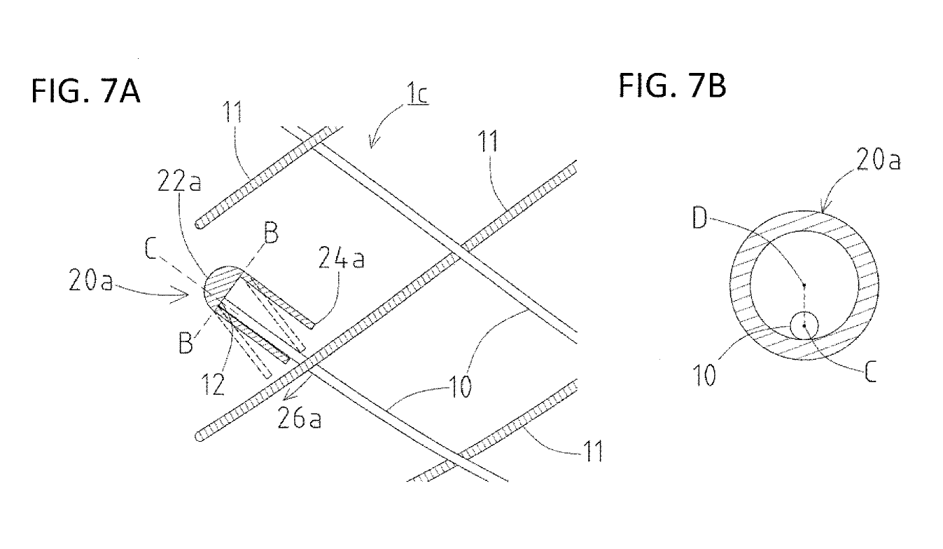

Next, a self-expandable stent 1c according to the disclosed embodiments is described with reference to FIGS. 7A and 7B. Note that FIG. 7A shows an enlarged view of a portion of the self-expandable stent 1c, and FIG. 7B is a cross-sectional view taken along line B-B of FIG. 7A (in other words, a view in a horizontal cross section orthogonal to an axis direction C of the plurality of first wires 10.).

As shown in FIG. 7A, in the self-expandable stent 1c, a first anchor member 20a is attached to the distal end 12 of the one of the plurality of first wires 10 as in the self-expandable stent 1. Here too, a distal end 22a of the first anchor member 20a is fixed to the distal end 12 of the one of the plurality of first wires 10, and a proximal end 24a is a free end that is not fixed to the one of the plurality of first wires 10.

As shown in FIG. 7B, a center C of the one of the plurality of first wires 10 is displaced (offset) from a center D of the first anchor member 20a. Therefore, the degree of freedom of the first anchor member 20a can be further increased. For example, the proximal end 24a can widely pivot about the distal end 22a in the direction of an arrow 26a like a pendulum. Therefore, even in a case where a blood vessel wall or digestive organ wall has an uneven surface due to the presence of a plaque or the like, contact can be made at the optimal position depending on the uneven surface. As a result, a risk can be further reduced that the self-expandable stent 1c will be axially or radially dislocated from the target site regardless of the conditions of a blood vessel wall or digestive organ wall at the target site.

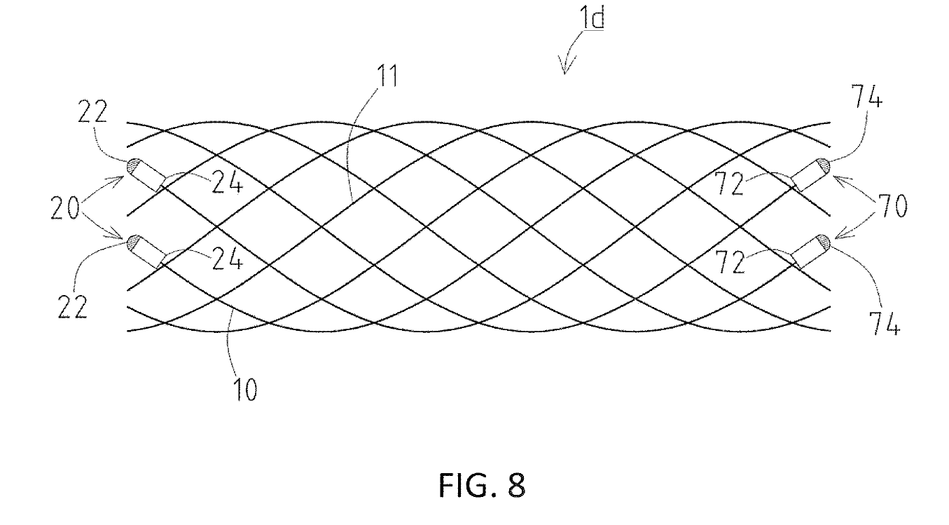

Finally, a self-expandable stent 1d according to the disclosed embodiments is described with reference to FIG. 8. Only differences from the self-expandable stent 1 shown in FIGS. 1 to 4 will be described.

As shown in FIG. 8, in the self-expandable stent 1d, a plurality of first anchor members 20 are attached to a corresponding number of distal ends 12 of the plurality of first wires 10, and a plurality of third anchor members 70 are attached to a corresponding number of proximal ends 14 of the plurality of first wires 10. The first anchor members 20 and the third anchor members 70 may be attached to the same or different ones of the plurality of first wires 10.

Proximal ends 74 of the third anchor members 70 are fixed to the proximal ends of the corresponding plurality of first wires 10, and distal ends 72 are free ends that are not fixed to the corresponding plurality of first wires 10. Therefore, the third anchor members 70 have a degree of freedom of motion with respect to the corresponding plurality of first wires 10 as in the first anchor members 20. For example, the proximal ends 72 can pivot about the distal ends 74 like a pendulum.

In the self-expandable stent 1d, the plurality of first anchor members 20 and the plurality of third anchor members are provided at the distal ends and the proximal ends of the plurality of first wires 10, respectively. Therefore, the anchor effect on a blood vessel wall or digestive organ wall can be improved, further reducing a risk that the self-expandable stent 1d will be axially or radially dislocated from the target site.

Note that the aforementioned self-expandable stents 1, 1a, 1b, 1c, and 1d each have a cylindrical shape with a constant outer diameter in the axial direction, but the configuration is not limited to this. For example, the self-expandable stents 1, 1a, 1b, 1c, and 1d may each have a small outer diameter at a central part and a large outer diameter at both ends. In other words, the self-expandable stents 1, 1a, 1b, 1c, and 1d may each have a flared distal end and flared proximal end.

As described above, in the self-expandable stents 1, 1a, 1b, 1c, and 1d, the first anchor member 20, 20a is attached to the distal end 12 of the one of the plurality of first wires 10. The distal end 22 or 22a of the first anchor member 20 or 20a is fixed to the one of the plurality of first wires 10, and the proximal end 24 or 24a is a free end that is not fixed to the one of the plurality of first wires 10. Therefore, when the self-expandable stents 1, 1a, 1b, 1c, and 1d are expanded at the target site, a gap is not easily formed between the first anchor member 20, 20a and a blood vessel wall or digestive organ wall. As a result, the anchor effect of the first anchor member 20, 20a on the blood vessel wall and digestive organ wall can be improved, reducing a risk that the self-expandable stents 1, 1a, 1b, 1c, and 1d will be axially or radially dislocated from the target site.

What is claimed is:

1. A self-expandable stent comprising:
a plurality of first wires;
a plurality of second wires interwoven with the plurality of first wires; and
a first anchor member attached to a distal end of one of the plurality of first wires,
wherein a distal end of the first anchor member is fixed to a distal end of the one of the plurality of first wires, and a proximal end of the first anchor member is not fixed to any of the plurality of first wires or to any of the plurality of second wires.

2. The self-expandable stent according to claim 1, further comprising:
a second anchor member attached to a distal end of one of the plurality of first wires that is different from the one of the plurality of first wires to which the first anchor member is attached, wherein:
a distal end of the second anchor member is fixed to the one of the plurality of first wires that is different from the one of the plurality of first wires to which the first anchor member is attached, and a proximal end of the second anchor member is not fixed to any of the plurality of first wires or to any of the plurality of second wires, and
the second anchor member is disposed at a position different from the first anchor member in an axial direction of the self-expandable stent.

3. The self-expandable stent according to claim 1, wherein a tensile strength of each of the plurality of first wires is higher than a tensile strength of each of the plurality of second wires.

4. The self-expandable stent according to claim 1, wherein a center of the one of the plurality of first wires is displaced from a center of the first anchor member, as viewed in a horizontal cross section orthogonal to an axial direction of the one of the plurality of first wires.

5. The self-expandable stent according to claim 1, wherein the plurality of first wires are made of a Co—Cr alloy.

6. The self-expandable stent according to claim 1, wherein the plurality of second wires are made of a Pt—Ni alloy.

7. The self-expandable stent according to claim 2, wherein a tensile strength of each of the plurality of first wires is higher than a tensile strength of each of the plurality of second wires.

8. The self-expandable stent according to claim 2, wherein a center of the one of the plurality of first wires is displaced from a center of the first anchor member, as viewed in a horizontal cross section orthogonal to an axial direction of the one of the plurality of first wires.

9. The self-expandable stent according to claim 2, wherein the plurality of first wires are made of a Co—Cr alloy.

10. The self-expandable stent according to claim 2, wherein the plurality of second wires are made of a Pt—Ni alloy.

11. The self-expandable stent according to claim 3, wherein a center of the at least one of the plurality of first wires is displaced from a center of the first anchor member, as viewed at a horizontal cross section orthogonal to the axial direction of the at least one of the plurality of first wires.

12. The self-expandable stent according to claim 3, wherein the plurality of first wires are made of a Co—Cr alloy.

13. The self-expandable stent according to claim 3, wherein the plurality of second wires are made of a Pt—Ni alloy.

14. The self-expandable stent according to claim 4, wherein the plurality of first wires are made of a Co—Cr alloy.

15. The self-expandable stent according to claim 4, wherein the plurality of second wires are made of a Pt—Ni alloy.

16. The self-expandable stent according to claim 1, further comprising:
a plurality of the first anchor members attached to distal ends of a corresponding number of the plurality of first wires,
wherein distal ends of the plurality of the first anchor members are fixed to distal ends of the corresponding number of plurality of first wires, and proximal ends of the first anchor members are not fixed to any of the plurality of first wires or to any of the plurality of second wires.

17. The self-expandable stent according to claim 16, wherein the plurality of the first anchor members are aligned with each other in an axial direction of the self-expandable stent.

18. The self-expandable stent according to claim 1, further comprising:
an additional anchor member attached to a proximal end of a same or different one of the plurality of first wires to which the first anchor member is attached,
wherein a proximal end of the additional anchor member is fixed to a proximal end of the same or different one of the plurality of first wires, and a distal end of the additional anchor member is not fixed to any of the plurality of first wires or to any of the plurality of second wires.

19. The self-expandable stent according to claim 1, wherein the first anchor member is a tube body.

20. The self-expandable stent according to claim 1, wherein the first anchor member is a hollow coil body.
